(19) United States
(12) Patent Application Publication
(10) Pub. No.: US 2024/0382163 A1
(43) Pub. Date: Nov. 21, 2024

(54) DEHYDRATION ESTIMATION DEVICE, ESTIMATION METHOD AND PROGRAM

(71) Applicant: Nippon Telegraph and Telephone Corporation, Tokyo (JP)

(72) Inventor: Yuki Hashimoto, Tokyo (JP)

(21) Appl. No.: 18/691,824

(22) PCT Filed: Oct. 25, 2021
(86) PCT No.: PCT/JP2021/039231

§ 371 Date: Mar. 13, 2024

(51) Int. Cl. A61B 5/00; A61B 5/01; A61B 5/024; A61B 5/145

(52) U.S. Cl. CPC A61B 5/7275; A61B 5/01; A61B 5/02405; A61B 5/14517; A61B 5/14546; A61B 5/6801

(57) ABSTRACT

A dehydration estimation device includes one or more processors and a storage device storing a program to be executed by the one or more processors, the program including instructions for estimating a blood electrolyte concentration of a measurement subject, and determining presence or absence of dehydration of the measurement subject on a basis of the blood electrolyte concentration.

### CROSS-REFERENCE TO RELATED APPLICATIONS

This application is a national phase entry of PCT Application No. PCT/JP2021/039231, filed on Oct. 25, 2021, which application is hereby incorporated herein by reference.

### TECHNICAL FIELD

The present invention relates to a dehydration estimation device, an estimation method, and a program capable of grasping a dehydration state of a person without blood sampling.

### BACKGROUND

In the human body, there are tissues that perform electrical activities such as muscles and nerves, and in order to continue to operate these tissues normally, a mechanism for keeping the electrolyte concentration in the body constant mainly by the functions of the automatic nerve system and the endocrine system is provided. For example, when a large amount of water in the body is lost due to long-time exposure to a hot environment or sweating due to excessive exercise or the like, and the electrolyte concentration in the body deviates from a normal value, various symptoms typified by dehydration and heat stroke occur.

The International Organization for Standardization (ISO) defines a limit value of a weight loss amount associated with sweating as an index for prevention of dehydration (see Non Patent Literature 1). However, in practice, the amount of electrolyte contained in sweat varies depending on individual differences and the presence or absence of heat acclimation. For this reason, the amount of water loss when the dehydration occurs differs for each individual, and also differs depending on the presence or absence of heat acclimation. Therefore, it can be said that monitoring the loss of water in the blood, the loss of electrolyte, and the electrolyte concentration in the blood is an effective means for grasping the dehydration state.

As a conventional method for measuring the electrolyte concentration in blood, there is a general biochemical test (see Non Patent Literature 2). The biochemical test is a test method for chemically analyzing and measuring blood components represented by electrolytes, enzymes, proteins, sugars, and lipids using sampled blood. This method is unsuitable for continuous measurement required for monitoring blood components because blood sampling is required.

### CITATION LIST

### Non Patent Literature

Non Patent Literature 1: “Shokuba no nechusho taisaku tsurezure kou (sono 2) (in Japanese) (Study on Heat Stroke Countermeasures in the Workplace (part 2))”, the National Institute of Occupational Safety and Health, Japan [searched on Aug. 10, 2021], <https://www.jniosh.johas.go.jp/publication/mail_mag/2013/61-column.html>

Non Patent Literature 2: “Seikagaku kensa (in Japanese) (Biochemical test)”, the National Hospital Organization Disaster Medical Center, [searched on Aug. 10, 2021], https://saigai.hosp.go.jp/kensaka/seikagakukensa.html

### SUMMARY

### Technical Problem

Embodiments of the present invention have been made to solve the above problems, and an object is to provide a dehydration estimation device, an estimation method, and a program capable of grasping a dehydration state of a person without blood sampling.

### Solution to Problem

A dehydration estimation device of the present invention includes: a blood electrolyte concentration estimation unit configured to estimate a blood electrolyte concentration of a measurement subject; and a dehydration determination unit configured to determine presence or absence of dehydration of the measurement subject on the basis of the blood electrolyte concentration.

In addition, a first configuration example of the dehydration estimation device of the present invention further includes: a sweat amount measurement unit configured to measure a sweat amount of the measurement subject; and a sweat electrolyte concentration measurement unit configured to measure a sweat electrolyte concentration of the measurement subject, in which the blood electrolyte concentration estimation unit estimates the blood electrolyte concentration on the basis of the sweat amount and the sweat electrolyte concentration.

In addition, the first configuration example of the dehydration estimation device of the present invention further includes: a sweat amount measurement unit configured to measure a sweat amount of the measurement subject; and a sweat electrolyte concentration estimation unit configured to estimate a sweat electrolyte concentration of the measurement subject on the basis of the sweat amount, in which the blood electrolyte concentration estimation unit estimates the blood electrolyte concentration on the basis of the sweat amount and the sweat electrolyte concentration.

In addition, the first configuration example of the dehydration estimation device of the present invention further includes: a heart rate measurement unit configured to measure a heart rate of the measurement subject; a temperature measurement unit configured to measure a temperature in the vicinity of the measurement subject; a humidity measurement unit configured to measure a humidity in the vicinity of the measurement subject; a sweat amount estimation unit configured to estimate a sweat amount of the measurement subject on the basis of measurement results of the heart rate measurement unit, the temperature measurement unit, and the humidity measurement unit; and a sweat electrolyte concentration estimation unit configured to estimate a sweat electrolyte concentration of the measurement subject on the basis of the sweat amount, in which the blood electrolyte concentration estimation unit estimates the blood electrolyte concentration on the basis of the sweat amount and the sweat electrolyte concentration.

In addition, the first configuration example of the dehydration estimation device of the present invention further includes: a water intake upper limit calculation unit configured to calculate an upper limit value of a normal range of water intake of the measurement subject on the basis of the sweat amount, the sweat electrolyte concentration, and the blood electrolyte concentration; and a dehydration predicted time calculation unit configured to predict a transition of a future blood electrolyte concentration on the basis of the sweat amount, the sweat electrolyte concentration, and the blood electrolyte concentration, and estimate a time until the blood electrolyte concentration reaches the upper limit value of the normal range.

In addition, in the first configuration example of the dehydration estimation device of the present invention, when the sweat amount is SW[t] and the sweat electrolyte concentration is CSW[t], the blood electrolyte concentration estimation unit estimates an intracellular fluid water amount VIC[t+Δt] at time t+Δt on the basis of an intracellular fluid water amount VIC[t] estimated immediately before for the measurement subject, estimates an extracellular fluid water mount VEC[t+Δt] at time t+Δt on the basis of an extracellular fluid water mount VEC[t] estimated immediately before for the measurement subject, the sweat amount SW[t], and a body surface area of the measurement subject, estimates an osmotic pressure CIC[t+Δt] of an intracellular fluid at time t+Δt on the basis of the intracellular fluid water amount VIC[t], an osmotic pressure CIC[t] of an intracellular fluid estimated immediately before for the measurement subject, and the intracellular fluid water amount VIC[t+Δt], and estimates a blood electrolyte concentration CEC[t+Δt] at time t+Δt on the basis of a blood electrolyte concentration CEC[t] estimated immediately before for the measurement subject, the extracellular fluid water mount VEC[t], the sweat electrolyte concentration CSW[t], the sweat amount SW[t], the body surface area, and the extracellular fluid water mount VEC[t+Δt].

In addition, a dehydration estimation method of embodiments of the present invention includes: a first step of measuring or estimating a sweat amount of a measurement subject; a second step of measuring or estimating a sweat electrolyte concentration of the measurement subject; a third step of estimating a blood electrolyte concentration of the measurement subject on the basis of the sweat amount and the sweat electrolyte concentration; and a fourth step of determining presence or absence of dehydration of the measurement subject on the basis of the blood electrolyte concentration.

In addition, a dehydration estimation program of embodiments of the present invention causes a computer to execute each of the above steps.

### Advantageous Effects of Embodiments of the Invention

According to embodiments of the present invention, by providing the blood electrolyte concentration estimation unit and the dehydration determination unit, it is possible to continuously estimate the blood electrolyte concentration of the measurement subject without blood sampling, and it is possible to grasp the dehydration state of the measurement subject on the basis of the blood electrolyte concentration.

### BRIEF DESCRIPTION OF THE DRAWINGS

FIG. 1 is a block diagram illustrating a configuration of a dehydration estimation device according to a first example of the present invention.

FIG. 2 is a flowchart describing an operation of the dehydration estimation device according to the first example of the present invention.

FIG. 3 is a block diagram illustrating a configuration of a dehydration estimation device according to a second example of the present invention.

FIG. 4 is a flowchart describing an operation of the dehydration estimation device according to the second example of the present invention.

FIG. 5 is a block diagram illustrating a configuration of a dehydration estimation device according to a third example of the present invention.

FIG. 6 is a flowchart describing an operation of the dehydration estimation device according to the third example of the present invention.

FIG. 7 is a block diagram illustrating a configuration of a sweat amount calculation unit of the dehydration estimation device according to the third example of the present invention.

FIG. 8 is a diagram illustrating a two-site two-layer model of the body of a measurement subject and a heat quantity flowing into and out of each site and each layer of the measurement subject.

FIG. 9 is a flowchart describing an operation of the sweat amount calculation unit of the dehydration estimation device according to the third example of the present invention.

FIG. 10 is a block diagram illustrating a configuration example of a computer that achieves the dehydration estimation device according to the first to third examples of the present invention.

### DETAILED DESCRIPTION OF ILLUSTRATIVE EMBODIMENTS

### Principle of Embodiments of the Invention

The present invention estimates an electrolyte concentration in blood of a measurement subject using a sweat amount of the measurement subject and an electrolyte concentration in sweat measured or estimated through a wearable sensor, and determines presence or absence of dehydration of the measurement subject on the basis of whether the blood electrolyte concentration is within a normal range.

Hereinafter, examples of the present invention will be described with reference to the drawings. In the following description, sodium is described as an example of the electrolyte in sweat and the electrolyte in blood, but other electrolytes contained in sweat may be the target.

### First Example

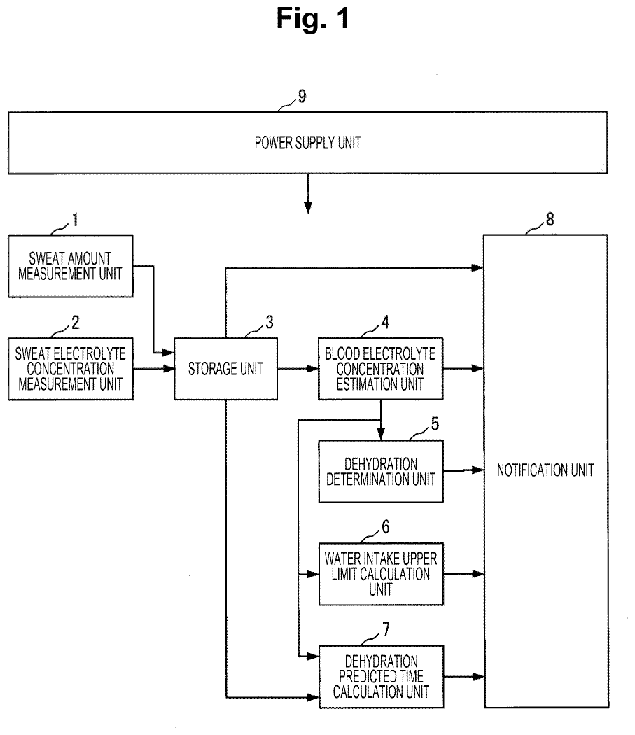

FIG. 1 is a block diagram illustrating a configuration of a dehydration estimation device according to a first example of the present invention. The dehydration estimation device includes a sweat amount measurement unit 1, a sweat electrolyte concentration measurement unit 2, a storage unit 3, a blood electrolyte concentration estimation unit 4, a dehydration determination unit 5, a water intake upper limit calculation unit 6, a dehydration predicted time calculation unit 7, a notification unit 8, and a power supply unit 9.

The sweat amount measurement unit 1 measures a sweat amount SW (volume of sweat secreted per unit area and per unit time) of the measurement subject. The device configuration of the sweat amount measurement unit 1 is disclosed in WO2021038742 and WO2021038758.

The sweat electrolyte concentration measurement unit 2 measures the electrolyte concentration in sweat of the measurement subject, specifically, a sodium concentration C_{SW}. The device configuration of the sweat electrolyte concentration measurement unit 2 is disclosed in WO2021038758.

The sweat amount measurement unit 1 and the sweat electrolyte concentration measurement unit 2 include a wearable sensor attached to the body of the measurement subject. In the technique disclosed in WO2021038742, the sweat amount of a measurement subject is calculated on the basis of characteristic of current application between electrodes of a wearable sensor. In the technique disclosed in WO2021038758, the sweat amount of a measurement subject and the electrolyte concentration in sweat of the measurement subject are calculated on the basis of light receiving characteristics of a light receiving element of a wearable sensor.

As disclosed in WO2021038742 and WO2021038758, the sweat amount measurement unit 1 and the sweat electrolyte concentration measurement unit 2 may include an analog front end (AFE) unit that amplifies a weak electric signal detected by a wearable sensor, and an analog digital converter (ADC) unit that converts the analog signal amplified by the AFE unit into digital data at a predetermined sampling frequency.

The storage unit 3 stores time-series data of the sweat amount SW measured by the sweat amount measurement unit 1 and time-series data of the sweat sodium concentration CSW measured by the sweat electrolyte concentration measurement unit 2. The storage unit 3 is achieved by nonvolatile memory represented by flash memory, volatile memory such as dynamic random access memory (DRAM), or the like.

The blood electrolyte concentration estimation unit 4 estimates a blood sodium concentration CEC of the measurement subject on the basis of the time-series data of the sweat amount SW and the time-series data of the sweat sodium concentration CSW stored in the storage unit 3.

Specifically, the blood electrolyte concentration estimation unit 4 estimates a blood sodium concentration C_{EC}[t+Δt] [mM] of the measurement subject after Δt according to Formula (1) on the basis of an estimated value C_{EC}[t] [mM] of the blood sodium concentration (sodium concentration in extracellular fluid) of the measurement subject at time t calculated before Δt, an estimated value V_{EC}[t] [L] of an extracellular fluid water mount of the measurement subject at time t, an estimated value V_{EC}[t+Δt] [L] of the extracellular fluid water amount of the measurement subject after Δt, a sweat sodium concentration C_{SW}[t] [mM] of the measurement subject at time t, a sweat amount SW[t] [L/m²/s] of the measurement subject at time t, and a body surface area S [m²] of the measurement subject.

$$\begin{matrix}Math . 1 & \\ {C}_{EC} [ t + Δ t ] = \frac{{C}_{EC} [ t ] \cdot {V}_{EC} [ t ] - {C}_{SW} [ t ] \cdot SW [ t ] \cdot S \cdot Δ t}{{V}_{EC} [ t + Δ t ]} & ( 1 )\end{matrix}$$

Δt is a calculation step time. The body surface area S [m²] of the measurement subject is a known value. The estimated value V_{EC}[t+Δt] [L] of the extracellular fluid water mount of the measurement subject at time t+Δt after Δt can be calculated according to Formula (2).

$$\begin{matrix}Math . 2 & \\ {V}_{EC} [ t + Δ t ] = {V}_{EC} [ t ] - SW [ t ] \cdot S \cdot Δ t + {V}_{i n} [ t ] & ( 2 )\end{matrix}$$

Vᵢₙ[t] [L] represents an estimated value of the amount of water transferred from the intracellular fluid to the extracellular fluid of the measurement subject at time t, and can be calculated according to Formula (3) described below.

$$\begin{matrix}Math . 3 & \\ {V}_{i n} [ t ] = \frac{( 2 {C}_{EC} [ t ] - {C}_{IC} [ t ] ) \cdot {V}_{EC} [ t ] + ( {C}_{IC} [ t ] - 2 {C}_{S W} [ t ] ) \cdot SW [ t ] \cdot S \cdot Δ t}{{C}_{IC} [ t ] \cdot {V}_{IC} [ t ] + 2 {C}_{EC} [ t ] \cdot {V}_{EC} [ t ] - 2 {C}_{SW} [ t ] \cdot SW [ t ] \cdot S \cdot Δ t} \cdot {V}_{IC} [ t ] & ( 3 )\end{matrix}$$

C_{IC}[t] [mOsm/L] represents an estimated value of the osmotic pressure of the intracellular fluid of the measurement subject at time t, and can be calculated according to Formula (4) described below.

$$\begin{matrix}Math . 4 & \\ {C}_{IC} [ t + Δ t ] = \frac{{C}_{IC} [ t ] \cdot {V}_{IC} [ t ]}{{V}_{IC} [ t + Δ t ]} & ( 4 )\end{matrix}$$

V_{IC}[t+Δt] [L] represents an estimated value of the intracellular fluid water amount of the measurement subject at time t+Δt, and can be calculated according to Formula (5) described below.

$$\begin{matrix}Math . 5 & \\ {V}_{IC} [ t + Δ t ] = {V}_{IC} [ t ] - {V}_{i n} [ t ] & ( 5 )\end{matrix}$$

C_{EC}[0] is an initial value of the blood sodium concentration of the measurement subject. For C_{EC}[0], it is sufficient if a practical value is set in advance as a known value obtained by past measurement.

V_{EC}[0] is an initial value of the extracellular fluid water mount of the measurement subject. V_{EC}[0] can be calculated according to, for example, Formula (6) described below.

$$\begin{matrix}Math . 6 & \\ {V}_{EC} [ 0 ] = \frac{W \cdot α \cdot β}{ρ} & ( 6 )\end{matrix}$$

W [kg] is a lean body mass of the measurement subject, ρ [kg/L] is the density of water, α is a variable corresponding to the sex and age of the measurement subject, and β is a ratio of the total amount of extracellular fluid to the total amount of water in the body of the measurement subject. For W, ρ, α, and β, it is sufficient if practical values are set in advance as a known value.

C_{IC}[0] is an initial value of the osmotic pressure of the intracellular fluid of the measurement subject. For C_{IC}[0], it is sufficient if a practical value is set in advance as a known value obtained by past measurement.

V_{IC}[0] is an initial value of the intracellular fluid water amount of the measurement subject, and can be calculated according to, for example, Formula (7) described below.

$$\begin{matrix}Math . 7 & \\ {V}_{IC} [ 0 ] = \frac{W \cdot α \cdot ( 1 - β )}{ρ} & ( 7 )\end{matrix}$$

In the initial calculation at time t=0, the blood electrolyte concentration estimation unit 4 estimates a water transfer amount Vᵢₙ[t]=Vᵢₙ[0] at time t=0 according to Formula (3) using an initial value V_{IC}[t]=V_{IC}[0] of the intracellular fluid water amount, an initial value C_{IC}[t]=C_{IC}[0] of the osmotic pressure of the intracellular fluid, an initial value C_{EC}[t]=C_{EC}[0] of the blood sodium concentration, an initial value V_{EC}[t]=V_{EC}[0] of the extracellular fluid water mount, a body surface area S, a sweat sodium concentration C_{SW}[t]=C_{SW}[0], and a sweat amount SW[t]=SW[0].

The blood electrolyte concentration estimation unit 4 estimates an intracellular fluid water amount V_{IC}[Δt] at time t=Δt after Δt according to Formula (5) using the initial value V_{IC}[t]=V_{IC}[0] of the intracellular fluid water amount and the water transfer amount Vᵢₙ[t]=Vᵢₙ[0]. In addition, the blood electrolyte concentration estimation unit 4 estimates an extracellular fluid water mount V_{EC}[Δt] at time t=Δt according to Formula (2) using the initial value V_{EC}[t]=V_{EC}[0] of the extracellular fluid water mount, the sweat amount SW[t]=SW[0], the body surface area S, and the water transfer amount Vᵢₙ[t]=Vᵢₙ[0].

Further, the blood electrolyte concentration estimation unit 4 estimates an osmotic pressure C_{IC}[Δt] of the intracellular fluid at time t=Δt according to Formula (4) using the initial value V_{IC}[t]=V_{IC}[0] of the intracellular fluid water amount, the initial value C_{IC}[t]=C_{IC}[0] of the osmotic pressure of the intracellular fluid, and the intracellular fluid water amount V_{IC}[Δt]. Then, the blood electrolyte concentration estimation unit 4 estimates a blood sodium concentration C_{EC}[Δt] at time t=Δt according to Formula (1) using the initial value C_{EC}[t]=C_{EC}[0] of the blood sodium concentration, the initial value V_{EC}[t]=V_{EC}[0] of the extracellular fluid water mount, the sweat sodium concentration C_{SW}[t]=C_{SW}[0], the sweat amount SW[t]=SW[0], the body surface area S, and the extracellular fluid water mount V_{EC}[Δt].

The blood electrolyte concentration estimation unit 4 can obtain time-series data of the blood sodium concentration C_{EC} by performing the same calculation for each cycle Δt thereafter. In the next calculation after Δt, it is sufficient if processing is performed by setting V_{IC}[Δt], V_{EC}[Δt], C_{IC}[Δt], and C_{EC}[Δt] calculated in the previous time to V_{IC}[t], V_{EC}[t], C_{IC}[t], and C_{EC}[t], respectively.

Note that when the measurement subject drinks water having a volume of V_{drink}[L] and a sodium concentration of C_{drink}[L] at time t, the calculation described below is performed. Specifically, it is sufficient if the blood electrolyte concentration estimation unit 4 uses Formula (8) instead of Formula (1), Formula (9) instead of Formula (2), and Formula (10) instead of Formula (3).

$$\begin{matrix}Math . 8 & \\ {C}_{EC} [ t + Δ t ] = \frac{{C}_{EC} [ t ] \cdot {V}_{EC} [ t ] - {C}_{SW} [ t ] \cdot SW [ t ] \cdot S \cdot Δ t + {C}_{drink} \cdot {V}_{drink}}{{V}_{EC} [ t + Δ t ]} & ( 8 )\end{matrix}$$

$$\begin{matrix}Math . 9 & \\ {V}_{EC} [ t + Δ t ] = {V}_{EC} [ t ] - SW [ t ] \cdot S \cdot Δ t + {V}_{drink} + {V}_{i n} [ t ] & ( 9 )\end{matrix}$$

$$\begin{matrix}Math . 10 & \\ {V}_{i n} [ t ] = ( \frac{\begin{matrix}( 2 {C}_{EC} [ t ] - {C}_{IC} [ t ] ) \cdot {V}_{EC} [ t ] + \\ ( {C}_{IC} [ t ] - 2 {C}_{SW} [ t ] ) \cdot SW [ t ] \cdot S \cdot Δ t\end{matrix}}{\begin{matrix}{C}_{IC} [ t ] \cdot {V}_{IC} [ t ] + 2 {C}_{EC} [ t ] \cdot {V}_{EC} [ t ] - \\ 2 {C}_{SW} [ t ] \cdot SW [ t ] \cdot S \cdot Δ t + 2 {C}_{drink} \cdot {V}_{drink}\end{matrix}} + \frac{2 {C}_{drink} \cdot {V}_{drink} - {C}_{IC} [ t ] \cdot {V}_{drink}}{\begin{matrix}{C}_{IC} [ t ] \cdot {V}_{IC} [ t ] + 2 {C}_{EC} [ t ] \cdot {V}_{EC} [ t ] - \\ 2 {C}_{SW} [ t ] \cdot SW [ t ] \cdot S \cdot Δ t + 2 {C}_{drink} \cdot {V}_{drink}\end{matrix}} ) \cdot {V}_{IC} [ t ] & ( 10 )\end{matrix}$$

However, in order to use Formulae (8) to (10), it is needless to say that the measurement subject or a third party needs to input values of the volume V_{drink} and the sodium concentration C_{drink} to the dehydration estimation device.

In addition, it is also possible to provide N (N is an integer of 2 or more) sensors in each of the sweat amount measurement unit 1 and the sweat electrolyte concentration measurement unit 2 and measure the sweat amount SW and the sweat sodium concentration C_{SW} at a plurality of points of the body of the measurement subject.

It is assumed that the sweat amount at a plurality of points of the body of the measurement subject at time t is SWᵢ[t] (i is an integer of 1 to N), that the sweat sodium concentration at the plurality of points is C_{SWi}[t], and that the body surface area of the point to be measured by each sensor is Sᵢ. A sum ΣSᵢ of Sᵢ is equal to the body surface area S of the measurement subject. The blood electrolyte concentration estimation unit 4 uses Formula (11) instead of Formula (1), Formula (12) instead of Formula (2), and Formula (13) instead of Formula (3).

$$\begin{matrix}Math . 11 & \\ {C}_{EC} [ t + Δ t ] = \frac{{C}_{EC} [ t ] \cdot {V}_{EC} [ t ] - {\sum }_{i}^{N} ( {C}_{SWi} [ t ] \cdot {SW}_{i} [ t ] \cdot {S}_{i} \cdot Δ t ) + {C}_{drink} {V}_{drink}}{{V}_{EC} [ t + Δ t ]} & ( 11 )\end{matrix}$$

$$\begin{matrix}Math . 12 & \\ {V}_{EC} [ t + Δ t ] = {V}_{EC} [ t ] - {\sum }_{i}^{N} ( {SW}_{i} [ t ] \cdot {S}_{i} \cdot Δ t ) + {V}_{drink} + {V}_{i n} [ t ] & ( 12 )\end{matrix}$$

$$\begin{matrix}Math . 13 & \\ {V}_{i n} [ t ] = ( \frac{\begin{matrix}2 {C}_{EC} [ t ] \cdot {V}_{EC} [ t ] - 2 {\sum }_{i}^{N} ( {C}_{SWi} [ t ] \cdot {SW}_{i} [ t ] \cdot {S}_{i} \cdot Δ t ) - \\ {C}_{IC} [ t ] \cdot {V}_{EC} [ t ] + {C}_{IC} [ t ] \cdot {\sum }_{i}^{N} ( {SW}_{i} [ t ] \cdot SW [ t ] \cdot S \cdot Δ t )\end{matrix}}{\begin{matrix}{C}_{IC} [ t ] \cdot {V}_{IC} [ t ] + 2 {C}_{EC} [ t ] \cdot {V}_{EC} [ t ] - \\ 2 {\sum }_{i}^{N} ( {C}_{SWi} [ t ] \cdot {SW}_{i} [ t ] \cdot {S}_{i} \cdot Δ t ) + 2 {C}_{drink} \cdot {V}_{drink}\end{matrix}} + \frac{2 {C}_{drink} \cdot {V}_{drink} - {C}_{IC} [ t ] \cdot {V}_{drink}}{\begin{matrix}{C}_{IC} [ t ] \cdot {V}_{IC} [ t ] + 2 {C}_{EC} [ t ] \cdot {V}_{EC} [ t ] - \\ 2 {\sum }_{i}^{N} ( {C}_{SWi} [ t ] \cdot {SW}_{i} [ t ] \cdot {S}_{i} \cdot Δ t ) + 2 {C}_{drink} \cdot {V}_{drink}\end{matrix}} ) \cdot {V}_{IC} [ t ] & ( 13 )\end{matrix}$$

The dehydration determination unit 5 uses the time-series data of the blood sodium concentration C_{EC} calculated by the blood electrolyte concentration estimation unit 4 as an input, and determines that there is no abnormality when the blood sodium concentration C_{EC}[t+Δt] at time t+Δt is within a predetermined normal range, and determines that dehydration is suspected when the blood sodium concentration C_{EC}[t+Δt] is outside the normal range. The dehydration determination unit 5 performs such determination of the presence or absence dehydration for each cycle Δt and outputs time-series data of the determination result.

The water intake upper limit calculation unit 6 calculates an upper limit value of the normal range of the water intake. Specifically, the water intake upper limit calculation unit 6 calculates a water intake upper limit value V_{d,tlv}[t] [L] at time t according to Formula (14) using the intracellular fluid water amount V_{IC}[t], the osmotic pressure C_{IC}[t] of the intracellular fluid, the extracellular fluid water mount V_{EC}[t], and the blood sodium concentration C_{EC}[t] calculated by the blood electrolyte concentration estimation unit 4. C_{EC,ltlv}[mM] is a lower limit value of the normal range of the blood sodium concentration, and is a known value.

$$Math . 14$$

$${V}_{d , tlv} [ t ] = ( \frac{{C}_{IC} [ t ]}{2 {C}_{EC , ltlv}} - 1 ) {V}_{IC} [ t ] + ( \frac{{C}_{EC} [ t ]}{{C}_{EC , ltlv}} - 1 ) {V}_{EC} [ t ] \qquad ( 14 )$$

In addition, the dehydration predicted time calculation unit 7 calculates, as the dehydration predicted time, the time until the blood sodium concentration C_{EC} reaches the upper limit value of the normal range when the measurement subject does not drink water. Specifically, the dehydration predicted time calculation unit 7 acquires, from the storage unit 3, the sweat sodium concentration C_{SW}[t] and the sweat amount SW[t] at the current time t at which the dehydration predicted time is to be calculated, and predicts the intracellular fluid water amount V_{IC}[t+Δt], the extracellular fluid water mount V_{EC}[t+Δt], the osmotic pressure C_{IC}[t+Δt] of the intracellular fluid, and the blood sodium concentration C_{EC}[t+Δt] at time t+Δt according to Formulae (5), (2), (4), (1), or Formulae (5), (12), (4), and (11), respectively.

Further, the dehydration predicted time calculation unit 7 predicts an intracellular fluid water amount V_{IC}[t+2Δt], an extracellular fluid water mount V_{EC}[t+2Δt], an osmotic pressure C_{IC}[t+2Δt] of the intracellular fluid, and a blood sodium concentration C_{EC}[t+2Δt] at time t+2Δt using the prediction results of VIC[t+Δt], V_{EC}[t+Δt], C_{IC}[t+Δt], and C_{EC}[t+Δt] one time before by setting C_{SW}[t] to the sweat sodium concentration C_{SW}[t+Δt] at time t+Δt and SW[t] to the sweat amount SW[t+Δt] at time t+Δt according to Formulae (5), (2), (4), (1), or (5), (12), (4), and (11), respectively. Similarly, values at times t+3Δt, t+4Δt, t+5Δt, . . . are predicted sequentially.

In this way, the dehydration predicted time calculation unit 7 sequentially predicts the value of the blood sodium concentration C_{EC} at each future time using the sweat sodium concentration C_{SW}[t] and the sweat amount SW [t] at the current time t as the sweat sodium concentration C_{SW} and the sweat amount SW at each future time. Then, it is sufficient if the dehydration predicted time calculation unit 7 estimates, as the dehydration predicted time, the time elapsed until the blood sodium concentration C_{EC} reaches an upper limit value C_{EC,utlv} of the normal range.

The notification unit 8 transmits the sweat amount SW measured by the sweat amount measurement unit 1, the sweat sodium concentration C_{SW} measured by the sweat electrolyte concentration measurement unit 2, the blood sodium concentration C_{EC} calculated by the blood electrolyte concentration estimation unit 4, the determination result by the dehydration determination unit 5, the water intake upper limit value V_{d,tlv} calculated by the water intake upper limit calculation unit 6, and the dehydration predicted time calculated by the dehydration predicted time calculation unit 7 to an external device (not illustrated) such as a smartphone in a wireless or wired manner.

Examples of the wireless communication standard include Bluetooth (registered trademark) Low Energy (BLE). In addition, examples of the wired communication standard include Ethernet (registered trademark).

The power supply unit 9 is a circuit that plays a role of supplying power to the entire dehydration estimation device.

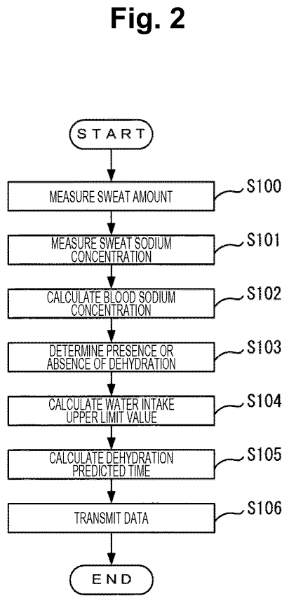

FIG. 2 is a flowchart describing an operation of the dehydration estimation device of the present example. The sweat amount measurement unit 1 measures the sweat amount SW[t] of the measurement subject at time t (step S100 in FIG. 2).

The sweat electrolyte concentration measurement unit 2 measures the sweat sodium concentration C_{SW}[t] of the measurement subject at time t (step S101 in FIG. 2).

The blood electrolyte concentration estimation unit 4 estimates the blood sodium concentration C_{EC}[t+Δt] of the measurement subject at time t+Δt (step S102 in FIG. 2).

The dehydration determination unit 5 determines the presence or absence of dehydration of the measurement subject on the basis of the blood sodium concentration C_{EC}[t+Δt] (step S103 in FIG. 2).

The water intake upper limit calculation unit 6 calculates the water intake upper limit value V_{d,tlv}[t] (step S104 in FIG. 2).

The dehydration predicted time calculation unit 7 calculates the dehydration predicted time (step S105 in FIG. 2).

The notification unit 8 transmits the sweat amount SW[t], the sweat sodium concentration C_{SW}[t], the blood sodium concentration C_{EC}[t+Δt], the determination result by the dehydration determination unit 5, the water intake upper limit value V_{d,tlv}[t], and the dehydration predicted time to an external device in a wireless or wired manner (step S106 in FIG. 2).

The dehydration estimation device performs the above processing of steps S100 to S106 for each cycle Δt.

Thus, in the present example, it is possible to continuously estimate the blood sodium concentration C_{EC} of the measurement subject without blood sampling, and it is possible to grasp the dehydration state of the measurement subject on the basis of the blood sodium concentration C_{EC}.

### Second Example

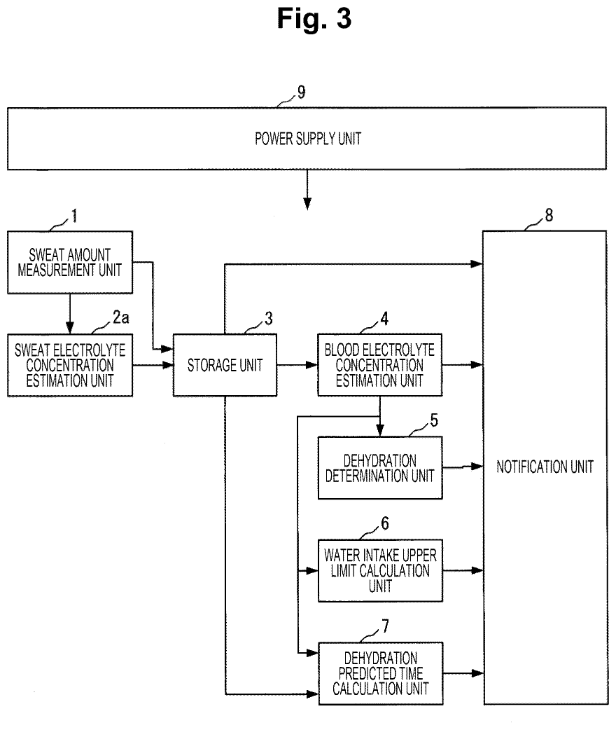

Next, a second example of the present invention will be described. FIG. 3 is a block diagram illustrating a configuration of a dehydration estimation device according to a second example of the present invention. The dehydration estimation device of the present example includes a sweat amount measurement unit 1, a sweat electrolyte concentration estimation unit 2a, a storage unit 3, a blood electrolyte concentration estimation unit 4, a dehydration determination unit 5, a water intake upper limit calculation unit 6, a dehydration predicted time calculation unit 7, a notification unit 8, and a power supply unit 9.

In the present example, the sweat electrolyte concentration estimation unit 2a is provided instead of the sweat electrolyte concentration measurement unit 2 of the first example.

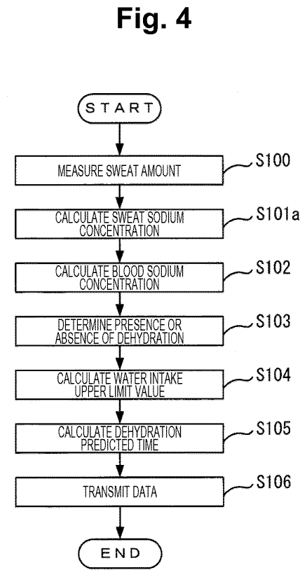

FIG. 4 is a flowchart describing an operation of the dehydration estimation device of the present example. The operation of the sweat amount measurement unit 1 (step S100 in FIG. 4) is the same as that in the first example.

The sweat electrolyte concentration estimation unit 2a estimates a sweat sodium concentration C_{SW}[t] of the measurement subject at time t according to Formula (15) described below using a sweat amount SW[t] of the measurement subject at time t (step S101a in FIGS. 4).

$${C}_{SW} [ t ] = SW [ t ] \cdot γ + δ \qquad ( 15 )$$

γ and δ are constants that vary depending on individual differences and the presence or absence of heat acclimation. It is sufficient if the relationship between the sweat amount and the sweat sodium concentration of the measurement subject is measured in advance and the constants γ and δ are set in advance.

As described in the first example, when the sweat amount SWᵢ[t] at a plurality of points of the body of the measurement subject is measured, it is sufficient if the calculation of Formula (15) is performed for each point of the body of the measurement subject to calculate the sweat sodium concentration C_{SWi}[t] at the plurality of points.

The operations of the blood electrolyte concentration estimation unit 4, the dehydration determination unit 5, the water intake upper limit calculation unit 6, the dehydration predicted time calculation unit 7, and the notification unit 8 (steps S102 to S106 in FIG. 4) are the same as those in the first example.

### Third Example

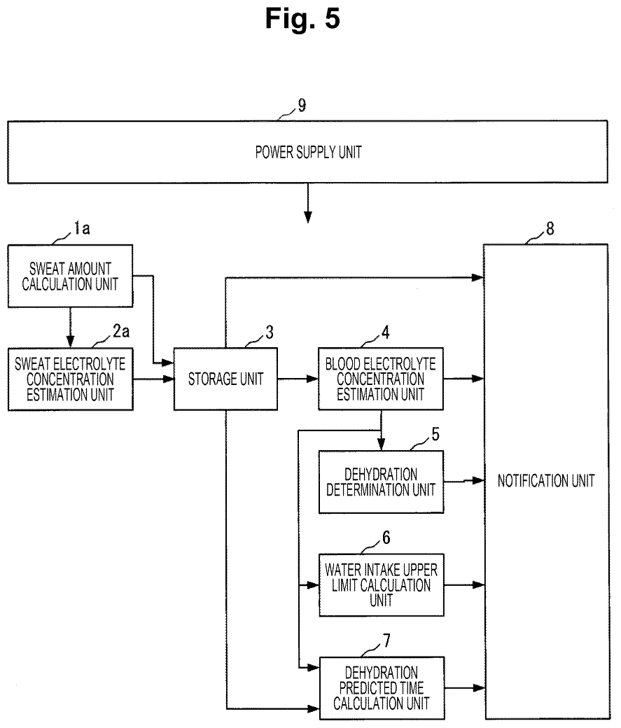

Next, a third example of the present invention will be described. FIG. 5 is a block diagram illustrating a configuration of a dehydration estimation device according to a third example of the present invention. The dehydration estimation device of the present example includes a sweat amount calculation unit 1a, a sweat electrolyte concentration estimation unit 2a, a storage unit 3, a blood electrolyte concentration estimation unit 4, a dehydration determination unit 5, a water intake upper limit calculation unit 6, a dehydration predicted time calculation unit 7, a notification unit 8, and a power supply unit 9.

In the present example, the sweat amount calculation unit 1a is provided instead of the sweat amount measurement unit 1 of the first example, and the sweat electrolyte concentration estimation unit 2a is provided instead of the sweat electrolyte concentration measurement unit 2.

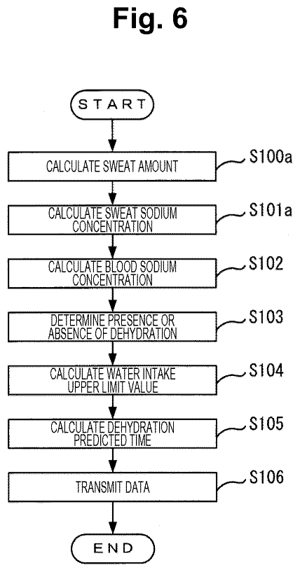

FIG. 6 is a flowchart describing an operation of the dehydration estimation device of the present example. The sweat amount calculation unit 1a calculates a sweat amount SW[t] of a measurement subject at time t (step S100a in FIG. 6).

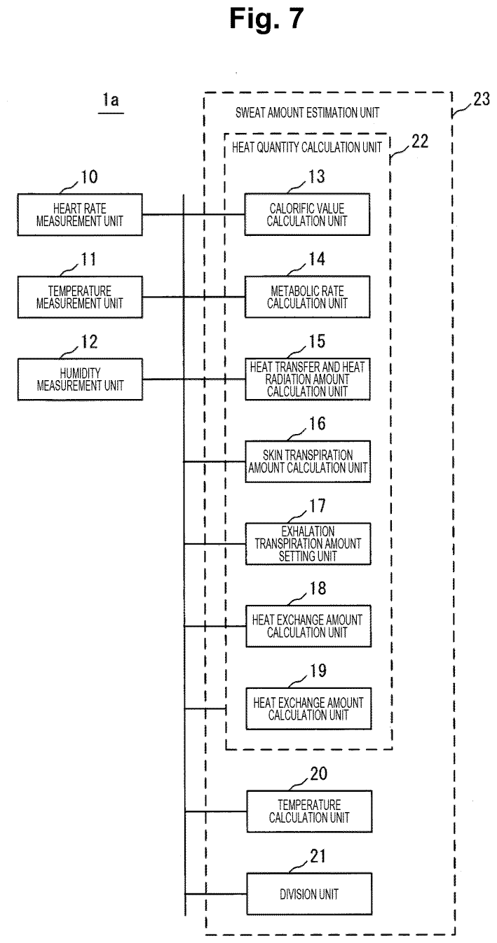

FIG. 7 is a block diagram illustrating a configuration of the sweat amount calculation unit 1a. The sweat amount calculation unit 1a includes a heart rate measurement unit 10, a temperature measurement unit 11, a humidity measurement unit 12, a calorific value calculation unit 13, a metabolic rate calculation unit 14, a heat transfer and heat radiation amount calculation unit 15, a skin transpiration amount calculation unit 16, an exhalation transpiration amount setting unit 17, a heat exchange amount calculation unit 18, a heat exchange amount calculation unit 19, a temperature calculation unit 20, and a division unit 21.

The calorific value calculation unit 13, the metabolic rate calculation unit 14, the heat transfer and heat radiation amount calculation unit 15, the skin transpiration amount calculation unit 16, the exhalation transpiration amount setting unit 17, the heat exchange amount calculation unit 18, and the heat exchange amount calculation unit 19 constitute a heat quantity calculation unit 22. The temperature calculation unit 20, the division unit 21, and the heat quantity calculation unit 22 constitute a sweat amount estimation unit 23.

The heart rate measurement unit 10 measures the heart rate or the pulse rate of the measurement subject. The heart rate measurement unit 10 includes, for example, a wear type or belt type electrocardiograph that measures the electrocardiogram of the measurement subject, and a calculation unit that calculates the heart rate from the electrocardiogram measured by the electrocardiograph. Alternatively, the heart rate measurement unit 10 includes a wristwatch-type or earphone-type pulse wave meter that measures a pulse wave of the measurement subject, and a calculation unit that calculates the heart rate (pulse rate) from the pulse wave measured by the pulse wave meter.

The temperature measurement unit 11 measures a temperature in the vicinity of the measurement subject (the air temperature of the atmosphere of the measurement subject). The temperature measurement unit 11 includes, for example, a thermometer. Alternatively, the temperature measurement unit 11 may acquire weather data in the vicinity of the measurement subject from an external weather system.

The humidity measurement unit 12 measures a humidity in the vicinity of the measurement subject (the humidity of the atmosphere of the measurement subject). The humidity measurement unit 12 includes, for example, a hygrometer. Alternatively, similarly to the temperature measurement unit 11, the humidity measurement unit 12 may acquire weather data in the vicinity of the measurement subject from an external weather system.

The heat quantity calculation unit 22 calculates a heat quantity flowing into and out of the deep parts and skin of the trunk (first site) and the limbs (second site) of the measurement subject on the basis of the heart rate of the measurement subject, the temperature in the vicinity of the measurement subject, and the humidity in the vicinity of the measurement subject.

The temperature calculation unit 20 calculates a skin temperature and a deep part temperature of each of the trunk and the limbs of the measurement subject on the basis of the heat quantity calculated by the heat quantity calculation unit 22.

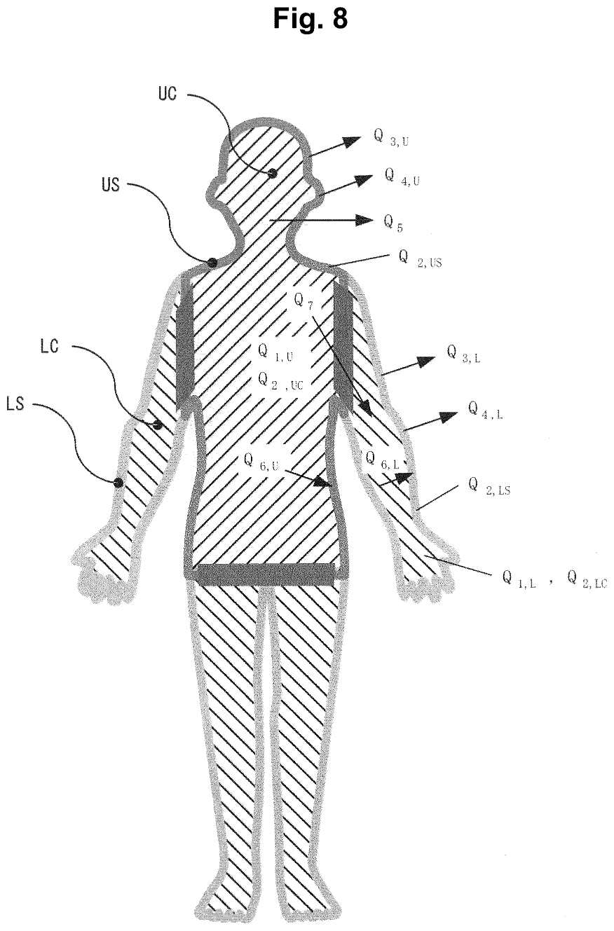

In the present example, as illustrated in FIG. 8, it is assumed that the body of the measurement subject includes two sites: a trunk U and limbs L, and the two sites: the trunk U and the limbs L each have two layers: a deep layer and a skin layer. That is, the body of the measurement subject includes a trunk deep layer UC, a trunk skin layer US, a limb deep layer LC, and a limb skin layer LS. In the present example, a heat quantity that flows into and out of each site and each layer of the measurement subject and changes from moment to moment is calculated on the basis of a temperature and a humidity to be acquired and heart rate information, a temperature change of each site and each layer of the measurement subject is estimated from a change in the heat quantity, and the skin transpiration amount of the measurement subject is determined to calculate the sweat amount.

In the present example, a method of calculating a deep body temperature change for each time step of acquiring sensor data will be described. In the present example, as described above, a calculation example in a case where the body of the measurement subject is regarded as including two sites: the trunk and the limbs is described, but the trunk may be replaced with the upper body and the limbs may be replaced with the lower body. That is, the body of the measurement subject may be regarded as including any two sites such as a first site and a second site.

Exemplary formulas for estimating a temperature change of the trunk skin layer US, a temperature change of the trunk deep layer UC, a temperature change of the limb skin layer LS, and a temperature change of the limb deep layer LC of the measurement subject are indicated according to Formulas (16) to (19), respectively. T_{US}[t] is the temperature [° C.] of the trunk skin layer US at time t, T_{UC}[t] is the temperature [° C.] of the trunk deep layer UC at time t, T_{LS}[t] is the temperature [° C.] of the limb skin layer LS at time t, and T_{LC}[t] is the temperature [° C.] of the limb deep layer LC at time t.

$$Math . 15$$

$${T}_{U S} [ t + Δ t ] = {T}_{U S} [ t ] + \frac{{Q}_{2 , US} - {Q}_{3 , U} - {Q}_{4 , U} + {Q}_{6 , U}}{W {C}_{U S}} Δ t \qquad ( 16 )$$

$$Math . 16$$

$${T}_{U C} [ t + Δ t ] = {T}_{U C} [ t ] + \frac{{Q}_{1 , U} + {Q}_{2 , U C} - {Q}_{5} - {Q}_{6 , U} - {Q}_{7}}{W {C}_{U C}} Δ t \qquad ( 17 )$$

$$Math . 17$$

$${T}_{L S} [ t + Δ t ] = {T}_{L S} [ t ] + \frac{{Q}_{2 , L S} - {Q}_{3 , L} - {Q}_{4 , L} + {Q}_{6 , L}}{W {C}_{L S}} Δ t \qquad ( 18 )$$

$$Math . 18$$

$${T}_{L C} [ t + Δ t ] = {T}_{L C} [ t ] + \frac{{Q}_{1 , L} + {Q}_{2 , LC} - {Q}_{6 , L} + {Q}_{7}}{W {C}_{L C}} Δ t \qquad ( 19 )$$

Q_{1,U} is a calorific value [W] of the trunk U due to exercise of the measurement subject, and Q_{1,L} is a calorific value [W] of the limbs L due to the exercise. Q_{2,US} is a metabolic rate [W] of the trunk skin layer US of the measurement subject, Q_{2,UC} is a metabolic rate [W] of the trunk deep layer UC, Q_{2,LS} is a metabolic rate [W] of the limb skin layer LS, and Q_{2,LC} is a metabolic rate [W] of the limb deep layer LC. Q_{3,U} (first heat exchange amount) is a heat transfer and heat radiation amount [W] between the skin and the outside air in the trunk U of the measurement subject, and Q_{3,L} (first heat exchange amount) is a heat transfer and heat radiation amount [W] between the skin and the outside air in the limbs L.

Q_{4,U} is a skin transpiration amount in the trunk U of the measurement subject, and Q_{4,L} is a skin transpiration amount in the limbs L. Q₅ (fourth heat exchange amount) is an exhalation transpiration amount [W] of the measurement subject. Q_{6,U} (second heat exchange amount) is a heat exchange amount [W] between the deep part and the skin in the trunk U of the measurement subject, and Q_{6,L} (second heat exchange amount) is a heat exchange amount [W] between the deep part and the skin in the limbs L. Q₇ (third heat exchange amount) is a heat exchange amount [W] between the trunk and the limbs in the deep part of the measurement subject.

WC_{US} is a heat capacity [J/° C.] of the trunk skin layer US of the measurement subject, WC_{UC} is a heat capacity [J/° C.] of the trunk deep layer UC, WC_{LS} is a heat capacity [J/° C.] of the limb skin layer LS, and WC_{LC} is a heat capacity [J/° C.] of the limb deep layer LC. Δt is a calculation step time, and is, for example, 1 [s] or less.

In addition, an average skin temperature Tₛₖ[t+Δt] and a deep body temperature T[t+Δt] at time t+Δt are indicated according to Formulae (20) and (21), respectively.

$$[ Math . 19 ]$$

$${T}_{sk} [ t + Δ t ] = sf\_conf \_US \times {T}_{US} [ t + Δ t ] + sf\_conf \_LS \times {T}_{LS} [ t + Δ t ] \qquad ( 20 )$$

$$Math . 20$$

$$T [ t + Δ t ] = {T}_{UC} [ t + Δ t ] \qquad ( 21 )$$

sf_conf_US is a proportion [%] of a surface area of the trunk U to the entire body surface of the measurement subject, and sf_conf_LS is a proportion [%] of a surface area of the limbs L to the entire body surface. The heat capacities WC_{US}, WC_{UC}, WC_{LS}, and WC_{LC}, and the proportions sf_conf_US and sf_conf_LS are known values, and it is sufficient if practical values are set in advance.

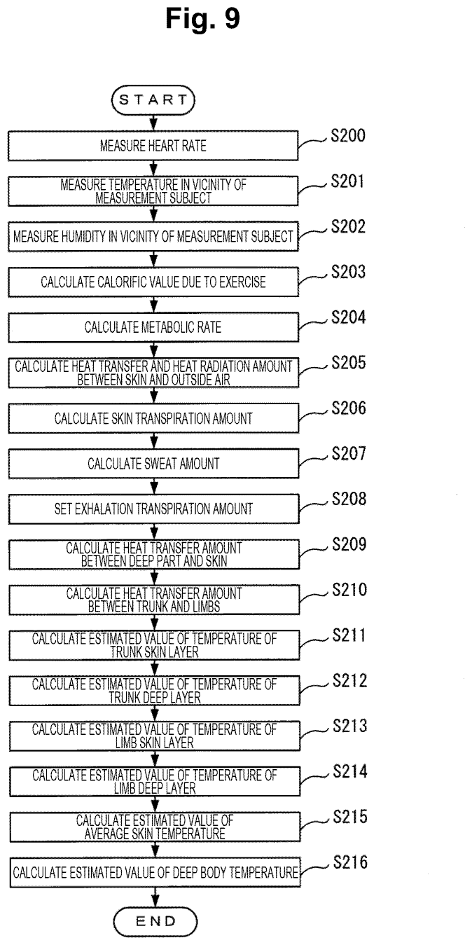

FIG. 9 is a flowchart describing an operation of the sweat amount calculation unit 1a of the present example. The heart rate measurement unit 10 measures the heart rate of the measurement subject (step S200 in FIG. 9). The temperature measurement unit 11 measures a temperature in the vicinity of the measurement subject (the air temperature of the atmosphere of the measurement subject) (step S201 in FIG. 9). The humidity measurement unit 12 measures a humidity in the vicinity of the measurement subject (the humidity of the atmosphere of the measurement subject) (step S202 in FIG. 9).

### Calculation of Calorific Value Q₁ Due to Exercise

Next, the calorific value calculation unit 13 calculates the calorific value Q_{1,U} [W] in the deep layer of the trunk U due to exercise of the measurement subject and the calorific value Q_{1,L} [W] in the deep layer of the limbs L due to the exercise according to Formulae (22) and (23), respectively, on the basis of the heart rate measured by the heart rate measurement unit 10 (step S203 in FIG. 9).

$$Math . 21$$

$${Q}_{1 , U} = ( METs [ t ] - 1 ) \times 1.05 \times weight \times 4184 \div 3600 \times ex\_conf \_U \qquad ( 22 )$$

$$Math . 22$$

$${Q}_{1 , L} = ( METs [ t ] - 1 ) \times 1.05 \times weight \times 4184 \div 3600 \times ex\_conf \_L \qquad ( 23 )$$

Formulae (22) and (23) are disclosed in the document ““Kenko dukuri no tameno undo shishin 2006˜Seikatu shukanbyo yobou no tameni˜(in Japanese) (Exercise Guidance for Health 2006˜For the Prevention of Lifestyle Diseases˜)”, the Ministry of Health, Labour and Welfare, 2006, <https://www.mhlw.go.jp/shingi/2006/07/dl/s0719-3c.pdf>”. weight is a weight [kg] of the measurement subject, ex_conf_U is a proportion [%] of the muscle mass of the trunk U to the whole body of the measurement subject, and ex_conf_L is a proportion [%] of the muscle mass of the limbs L to the whole body. The weight weight and the proportions ex_conf_U and ex_conf_L are known values, and it is sufficient if practical values are set in advance.

In addition, METs[t] represents Mets. It is sufficient if the calorific value calculation unit 13 calculates METs[t] according to any one of Formula (24) or (25) using a heart rate HR[t] [bpm], a resting heart rate HRᵣₑₛₜ, and a maximum heart rate HRₘₐₓ at time t acquired by the heart rate measurement unit 10.

$$Math . 23$$

$$METs [ t ] = \frac{HR [ t ] - {HR}_{rest}}{{HR}_{max} - {HR}_{rest}} \times 15 \frac{{HR}_{max}}{{HR}_{rest}} \div 3.5 \qquad ( 24 )$$

$$[ Math . 24 ]$$

$$METs [ t ] = 6.1 ( HR [ t ] - {HR}_{rest} ) - ( 6.1 - 1 ) \qquad ( 25 )$$

Formula (25) is disclosed in the document “J. R. Wicks, et al., “HR Index-A Simple Method for the Prediction of Oxygen Uptake”, Medicine and Science in Sports and Exercise, 2011”.

For the resting heart rate HRᵣₑₛₜ and the maximum heart rate HRₘₐₓ, it is sufficient if practical values are used as known values obtained by past measurement and the resting heart rate HRᵣₑₛₜ and the maximum heart rate HRₘₐₓ are appropriately set in performing calculation. In addition, not only the instantaneous value but also the time average value (for example, an average value for six minutes) may be used for METs[t] used in Formulae (22) and (23).

### Calculation of Metabolic Rate Q₂

Next, the metabolic rate calculation unit 14 calculates the metabolic rate Q_{2,US} [W] of the trunk skin layer US, the metabolic rate Q_{2,UC} [W] of the trunk deep layer UC, the metabolic rate Q_{2,LS} [W] of the limb skin layer LS, and the metabolic rate Q_{2,LC} [W] of the limb deep layer LC of the measurement subject as indicated according to Formulae (26) to (29), respectively, on the basis of an estimated value Tsk[t] [° C.] of an average skin temperature of the measurement subject and an estimated value T[t] [° C.] of the deep body temperature at time t calculated before Δt by the temperature calculation unit 20 (step S204 in FIG. 9).

$$Math . 25$$

$${Q}_{2 , US} = ( volume\_US \times {A}_{skin} ) \times {1.1}^{{T}_{sk} [ t ] - {T}_{sk} [ 0 ]} \qquad ( 26 )$$

$$Math . 26$$

$${Q}_{2 , UC} = ( \frac{weight\_U}{weight\_U + weight\_L} \times M - volume\_US \times {A}_{skin} ) \times {1.1}^{T [ t ] - T [ 0 ]} \qquad ( 27 )$$

$$Math . 27$$

$${Q}_{2 , LS} = ( volume\_LS \times {A}_{skin} ) \times {1.1}^{{T}_{sk} [ t ] - {T}_{sk} [ 0 ]} \qquad ( 28 )$$

$$Math . 28$$

$${Q}_{2 , LC} = ( \frac{weight\_L}{weight\_U + weight\_L} \times M - volume\_LS \times {A}_{skin} ) \times {1.1}^{T [ t ] - T [ 0 ]} \qquad ( 29 )$$

Formulae (26) to (29) are disclosed in the document “Ronald J Spiegel, “A Review of Numerical Models for Predicting the Energy Deposition and Resultant Thermal Response of Humans Exposed to Electromagnetic Fields”, IEEE Transactions on Microwave Theory and Techniques, Volume 32, Issue 8, 1984”. Aₛₖᵢₙ is a constant related to metabolism, volume_US is a volume [m³] of the trunk skin layer US of the measurement subject, volume_LS is a volume [m³] of the limb skin layer LS, weight_U is a weight [kg] of the trunk U of the measurement subject, and weight_L is a weight [kg] of the limbs L. The constant Aₛₖᵢₙ, the volumes volume_US and volume_LS, and the weights weight_U and weight_L are known values, and it is sufficient if practical values are set in advance.

Tₛₖ[0] is an initial value at the time of calculation of the average skin temperature Tₛₖ[t], and T[0] is an initial value at the time of calculation of the deep body temperature T[t]. In the present example, as an initial value T_{US}[0] of the temperature of the trunk skin layer US, an initial value T_{UC}[0] of the temperature of the trunk deep layer UC, an initial value T_{LS}[0] of the temperature of the limb skin layer LS, and an initial value T_{LC}[0] of the temperature of the limb deep layer LC, actual measured values measured by another temperature measurement device or practical values such as general resting known values are used. Therefore, in the first calculation of the metabolic rate Q₂ at time t=0, Tₛₖ[t]=Tₛₖ[0].

Similarly, as the initial value T[0] of the deep body temperature T[t], an actual measured value measured by another temperature measurement device or a practical value such as a general resting known value is used. Therefore, in the first calculation of the metabolic rate Q₂ at time t=0, T[t]=T[0].

In addition, the metabolic rate calculation unit 14 calculates M in Formulae (27) and (29) as indicated by Formula (30).

$$Math . 29$$

$$M = \text{} ( 0.1238 + 0.0481 \times weight\_C + \text{} 0.0234 \times height \text{} - \text{} 0.0138 \times \qquad ( 30 )$$

$$age \text{} - \text{} sexcoef ) \times \text{} {10}^{6} \times \text{} 24 \div 3600 \times activity\_level \times {A}_{coef}$$

Formula (30) is disclosed in the document “AA Ganpule, et al., “Interindividual variability in sleeping metabolic rate in Japanese subjects”, European Journal of Clinical Nutrition, volume 61, 2007”. weight_C is a weight [kg] of the deep part of the measurement subject, height is a height [cm] of the measurement subject, and age is an age of the measurement subject. sexcoef is a constant of 0.5473 in a case where the measurement subject is a male and 0.5473×2 in a case where the measurement subject is a female. activity_level is a physical activity level, and A_{coef} is a parameter for metabolic adjustment. The weight weight_C, the height height, the age age, the constants sexcoef and activity_level, and the parameter A_{coef} are known values, and it is sufficient if practical values are set in advance.

As an example of setting activity_level, it is sufficient if activity_level is set to about 1.5 in a case where most of the life of the measurement subject is sedentary and usually performs static activity. In a case where the measurement subject usually performs work in a sitting position, but includes movement in the workplace, work in a standing position, serving a customer, or the like, or includes any of commuting, shopping, housework, light sports, and the like, it is sufficient if activity_level is set to about 1.75. In a case where the measurement subject is a worker who moves or stands up a lot, or has an active exercise habit in leisure such as sports, it is sufficient if activity_level is set to about 2.0. As described above, it is sufficient if activity_level is appropriately set according to an exercise situation of the measurement subject.

### Calculation of Heat Transfer and Heat Radiation Amount Q₃ between Skin and Outside Air

Next, the heat transfer and heat radiation amount calculation unit 15 calculates the heat transfer and heat radiation amount Q_{3,U} [W] between the skin and the outside air in the trunk U and the heat transfer and heat radiation amount Q_{3,L} [W] between the skin and the outside air in the limbs L of the measurement subject on the basis of a temperature Tₐ[t] [C] in the vicinity of the measurement subject measured by the temperature measurement unit 11, and the estimated value Tₛₖ[t] [° C.] of the average skin temperature of the measurement subject, the estimated value T_{US}[t] [° C.] of the temperature of the trunk skin layer US, and the estimated value T_{LS}[t] [° C.] of the temperature of the limb skin layer LS at time t, which are calculated before Δt by the temperature calculation unit 20 (step S205 in FIG. 9). In a case where the temperature measurement unit 11 measures an air temperature Tₐ[t] [C] outside the clothing of the measurement subject, the heat transfer and heat radiation amounts Q_{3,U} and Q_{3,L} are indicated according to Formulae (31) and (32).

$$Math . 30$$

$${Q}_{3 , U} = sf\_conf \_US \times fcl\_US \times HS \times ( {T}_{US} [ t ] - {T}_{a} [ t ] \qquad ( 31 )$$

$$Math . 31$$

$${Q}_{3 , L} = \text{} sf\_conf \_LS \times ( fcl\_LS \times coverage + 1 - coverage ) \times HS \times ( {T}_{LS} [ t ] - {T}_{a} [ t ] ) \qquad ( 32 )$$

The proportions sf_conf_US and sf_conf_LS are as described above. fcl_US is a constant representing the heat transfer efficiency of the trunk U by the clothing, fcl_LS is a constant representing the heat transfer efficiency of the limbs L by the clothing, and coverage is a proportion [%] at which the limbs L are covered with the clothing. The proportions sf_conf_US and sf_conf_LS, the constants fcl_US and fcl_LS, and the proportion coverage are known values, and it is sufficient if practical values are set in advance. It is sufficient if the constants fcl_US and fcl_LS, and the proportion coverage are appropriately set according to the clothes of the measurement subject.

HS is a heat exchange coefficient [W/° C.] of the measurement subject with the air. The heat transfer and heat radiation amount calculation unit 15 calculates the heat exchange coefficient HS [W/° C.] according to Formula (33).

$$[ Math . 32 ]$$

$$HS = ( {H}_{cm} + {H}_{r} ) \times sf \qquad ( 33 )$$

sf is a body surface area [m²] of the measurement subject. The heat transfer and heat radiation amount calculation unit 15 can calculate the body surface area sf [m²] from the weight weight [kg] and the height height [m] of the measurement subject. Examples of the calculation formula of the body surface area sf [m²] include estimation formulas such as DuBois's formula and Fujimoto's formula.

H_{cm} is a convective heat transfer coefficient [W/° C./m²] and Hᵣ is a radiative heat transfer coefficient [W/° C./m²]. The heat transfer and heat radiation amount calculation unit 15 calculates the convective heat transfer coefficient H_{cm} [W/° C./m²] and the radiative heat transfer coefficient Hᵣ [W/° C./m²] according to Formulas (34) and (35), respectively.

$$Math . 33$$

$${H}_{cm} = \sqrt{4.63 \times \sqrt{\vert {T}_{sk} [ t ] - {T}_{a} [ t ] \vert} + 199.74 \times {V}_{air} - 9.8} \qquad ( 34 )$$

$$[ Math . 34 ]$$

$${H}_{r} = ( 5.67 \times {10}^{- 8} ) \times 0.99 \times 0.93 \times 0.794 \times \{ {( {T}_{sk} [ t ] + 273 )}^{2} + {( {T}_{a} [ t ] + 273 )}^{2} \} \times \{ ( {T}_{sk} [ t ] + 273 ) + ( {T}_{a} [ t ] + 273 ) \} \qquad ( 35 )$$

Formulae (34) and (35) are disclosed in the document “D. Fiala, et al., “A computer model of human thermoregulation for a wide range of environmental conditions: the passive system”, Journal of Applied Physiology, 1985”. Vₐᵢᵣ is a wind speed [m/s]. The heat transfer and heat radiation amount calculation unit 15 may use, as the wind speed Vₐᵢᵣ [m/s], an actual measured value measured by an anemometer or the like, or a known value of a wind speed in the clothing disclosed in documents or the like.

When the temperature measurement unit 11 measures the temperature Tₐ[t] [° C.] in the clothing of the measurement subject, the heat transfer and heat radiation amount calculation unit 15 calculates the heat transfer and heat radiation amounts Q_{3,U} and Q_{3,L} according to Formulae (36) and (37).

$$Math . 35$$

$${Q}_{3 , U} = sf\_conf \_US \times \frac{HS}{fcl\_US} \times ( {T}_{US} [ t ] - {T}_{a} [ t ] ) \qquad ( 36 )$$

$$Math . 36$$

$${Q}_{3 , L} = sf\_conf \_LS \times \frac{HS}{fcl\_US} \times ( {T}_{LS} [ t ] - {T}_{a} [ t ] ) \qquad ( 37 )$$

### Calculation of Skin Transpiration Amount Q₄

Next, the skin transpiration amount calculation unit 16 calculates the skin transpiration amount Q_{4,U} [W] in the trunk U and the skin transpiration amount Q_{4,L} [W] in the limbs L of the measurement subject according to Formulae (38) and (39), respectively, on the basis of the temperature Tₐ[t] [° C.] in the vicinity of the measurement subject measured by the temperature measurement unit 11, a relative humidity humidity [t] [%] in the vicinity of the measurement subject measured by the humidity measurement unit 12, and the estimated value Tₛₖ[t] [° C.] of the average skin temperature and the estimated value T[t] [° C.] of the deep body temperature at time t calculated before Δt by the temperature calculation unit 20 (step S206 in FIG. 9).

$$Math . 37$$

$${Q}_{4 , U} = sw\_conf \_US \times swv \qquad ( 38 )$$

$$Math . 38$$

$${Q}_{4 \_L} = sw\_conf \_LS \times swv \qquad ( 39 )$$

sw_conf_US is a proportion [%] of a sweat amount of the trunk U to the entire body surface of the measurement subject, and sw_conf_LS is a proportion [%] of a sweat amount of the limbs L to the entire body surface. The proportions sw_conf_US and sw_conf_LS are known values, and it is sufficient if practical values are set in advance. swv is a skin transpiration amount [W] of the whole body of the measurement subject. The skin transpiration amount calculation unit 16 may calculate the skin transpiration amount swv [W] according to Formula (40).

$$Math . 39$$

$$swv = min ( E , {E}_{max} ) \qquad ( 40 )$$

min (E,Eₘₐₓ) means that a smaller one of E and Eₘₐₓ is employed. E is a sum [W] of insensible transpiration and sensible transpiration in the skin of the measurement subject. The skin transpiration amount calculation unit 16 may calculate the sum E [W] of insensible transpiration and sensible transpiration according to Formula (41).

$$Math . 40$$

$$E = P I + {Q}_{e v} \times swrate \div 60 \qquad ( 41 )$$

PI is insensible transpiration [W] in the skin of the measurement subject, and Qₑᵥ is evaporation heat [J/g] of water. The insensible transpiration PI [W] and the evaporation heat Qₑᵥ [J/g] are known values, and it is sufficient if practical values are set in advance. swrate is sensible transpiration [g/min]. The skin transpiration amount calculation unit 16 may calculate the sensible transpiration swrate [g/min] according to Formula (42).

$$\begin{matrix}Math . 41 & \\ swrate = ( a11 \times tanh ( b 11 \times ( {T}_{s k} [ τ ] - {T}_{sk} [ 0 ] ) - b 10 ) + a 10 ) \times ( {T}_{s k} [ t ] - {T}_{s k} [ 0 ] ) + ( a 21 \times tanh ( b 21 \times ( T [ t ] - T [ 0 ] ) - b 20 ) + a 20 ) \times ( T [ t ] - T [ 0 ] ) & ( 42 )\end{matrix}$$

Formula (42) is disclosed in the document “D. Fiala, et al., “Computer prediction of human thermoregulatory and temperature responses to a wide range of environmental conditions”, International Journal of Biometeorology, volume 45, 2001”. aij and bij (i=1 and 2,and j=0 and 1) are sweating coefficients. Each of the sweating coefficients aij and bij is a known value, and it is sufficient if practical values are set in advance according to the ease of sweating of the measurement subject. Specifically, it is sufficient if the sweating coefficients aij and bij are set as indicated according to Formula (43) according to three levels: low (hard to sweat), normal (normal), and high (easy to sweat).

$$\begin{matrix}Math . 42 & \\ [ \begin{matrix}a 10 & a 11 & a 20 & a 21 & b 10 & b 11 & b 20 & b 21\end{matrix} ] = \{ \begin{matrix}[ 0.95 & 0.55 & 3.8 & 3.2 & 0.09 & 0.59 & 1.8 & 2.7 ] & ( low ) \\ [ 1.2 & 0.8 & 6.3 & 5.7 & 0.19 & 0.59 & 1.03 & 1.98 ] & ( normal ) \\ [ 1.35 & 0.95 & 7.3 & 6.7 & 0.15 & 0.59 & 0.47 & 2.3 ] & ( high )\end{matrix} & ( 43 )\end{matrix}$$

On the other hand, Eₘₐₓ is a maximum evaporation heat [W]. In a case where the temperature measurement unit 11 measures the air temperature Tₐ[t] [° C.] outside the clothing of the measurement subject and the humidity measurement unit 12 measures the relative humidity humidity [t] [%] outside the clothing of the measurement subject, the skin transpiration amount calculation unit 16 can calculate the maximum evaporation heat Eₘₐₓ [W] according to Formula (44).

$$\begin{matrix}Math . 43 & \\ {E}_{max} = {E}_{max \_ coef} \times {H}_{c} \times ( {P}_{s} - humidity [ t ] \times {P}_{a} ) \times ( sw\_conf \_US \times fpcl\_US + sw\_conf \_LS \times ( fpcl\_LS \times coverage + 1 - coverage ) ) & ( 44 )\end{matrix}$$

In addition, in a case where the temperature measurement unit 11 measures the temperature Tₐ[t] [° C.] in the clothing of the measurement subject and the humidity measurement unit 12 measures the relative humidity humidity [t] [%] in the clothing of the measurement subject, the skin transpiration amount calculation unit 16 can calculate the maximum evaporation heat Eₘₐₓ [W] according to Formula (45).

$$\begin{matrix}Math . 44 & \\ {E}_{max} = {E}_{max \_ coef} \times {H}_{c} \times ( {P}_{s} - fpcl\_US \times humidity [ t ] \times {P}_{a} ) \times ( sw\_conf \_US \times fpcl\_US + sw\_conf \_LS \times ( fpcl\_LS \times coverage + 1 - coverage ) ) & ( 45 )\end{matrix}$$

The proportions sw_conf_US, sw_conf_LS, and coverage are as described above. fpcl_US is a constant representing a heat transfer efficiency of the trunk U of the measurement subject by the clothing, fpcl_LS is a constant representing a heat transfer efficiency of the limbs L by the clothing, and E_{max_coef} is a constant related to the maximum evaporation heat. The constants fpcl_US, fpcl_LS, and E_{max_coef} are known values, and it is sufficient if practical values are set in advance.

H_{c} is heat transfer [W·m²/° C.] by convection depending on the wind speed of the air. The skin transpiration amount calculation unit 16 can calculate the heat transfer H_{c} [W·m²/C] according to Formula (46).

$$\begin{matrix}Math . 45 & \\ {H}_{c} = 3.0 \times \sqrt{10 \times {V}_{air}} & ( 46 )\end{matrix}$$

Formulae (44) and (46) are disclosed in the document “I. Laakso, et al., “Dominant factors affecting temperature rise in simulations of human thermoregulation during RF exposure”, Physics in Medicine and Biology, Volume 56, 2011”. Pₛ is a saturated water vapor pressure [kPa] in the skin layer of the measurement subject. The skin transpiration amount calculation unit 16 can calculate the saturated water vapor pressure Pₛ [kPa] according to Formula (47).

$$\begin{matrix}Math . 46 & \\ {P}_{s} = 7.5 \times 1 {0}^{7.23 - \frac{1750.3}{273 + {T}_{sk} [ t ] - 38.1}} & ( 47 )\end{matrix}$$

Pₐ is a saturated water vapor pressure [kPa] in the atmosphere in which the humidity is measured. The skin transpiration amount calculation unit 16 may calculate the saturated water vapor pressure Pₐ [kPa] according to Formula (48).

$$\begin{matrix}Math . 47 & \\ {P}_{a} = 7.5 \times 1 {0}^{7.23 - \frac{1750.3}{273 + {T}_{a} [ t ] - 38.1}} & ( 48 )\end{matrix}$$

### Calculation of Sweat Amount SW

Next, the division unit 21 calculates the sweat amount SW[t] of the measurement subject at time t by dividing the skin transpiration amount swv calculated by the skin transpiration amount calculation unit 16 by the body surface area S of the measurement subject (step S207 in FIG. 9).

$$S W [ t ] = swv / S \qquad ( 49 )$$

As described above, the sweat amount calculation unit 1a can calculate the sweat amount SW[t] of the measurement subject. However, in order to calculate the sweat amount SW[t], it is necessary to calculate the skin transpiration amount swv, and in order to calculate the skin transpiration amount swv, the estimated value Tₛₖ[t] of the average skin temperature and the estimated value T[t] of the deep body temperature at time t, which are calculated before Δt by the temperature calculation unit 20, are necessary, so that the processing of steps S208 to S216 described below is required.

### Determination of Exhalation Transpiration Amount Q₅

The exhalation transpiration amount setting unit 17 sets a transpiration amount Q₅ [W] by the exhalation of the measurement subject as indicated according to Formula (50) (step S208 in FIG. 9). The exhalation transpiration amount Q₅ [W] corresponds to a heat exchange amount between the outside air and the deep part of the measurement subject.

$$\begin{matrix}Math . 48 & \\ {Q}_{5} = 9 \text{.54} & ( 50 )\end{matrix}$$

### Calculation of Heat Exchange Amount Q₆ between Deep Part and Skin

Next, the heat exchange amount calculation unit 18 calculates the heat exchange amount Q_{6,U} [W] between the deep part and the skin in the trunk U of the measurement subject and the heat exchange amount Q_{6,L} [W] between the deep part and the skin in the limbs L according to Formulae (51) and (52), respectively, on the basis of the heart rate HR[t] [bpm] measured by the heart rate measurement unit 10, and the estimated value T_{US}[t] [° C.] of the temperature of the trunk skin layer US, the estimated value T_{UC}[t] [° C.] of the temperature of the trunk deep layer UC, the estimated value T_{LS}[t] [° C.] of the temperature of the limb skin layer LS, the estimated value T_{LC}[t] [° C.] of the temperature of the limb deep layer LC, the estimated value Tₛₖ[t] [° C.] of the average skin temperature, and the estimated value T[t] [° C.] of the deep body temperature at time t, calculated before Δt by the temperature calculation unit 20 (step S209 in FIG. 9).

$$\begin{matrix}Math . 49 & \\ {Q}_{6 , U} = sf\_conf \_US \times hx \times ( {T}_{U C} [ t ] - {T}_{U S} [ t ] ) & ( 51 )\end{matrix}$$

$$\begin{matrix}Math . 50 & \\ {Q}_{6 , L} = sf\_conf \_LS \times hx \times ( {T}_{L C} [ t ] - {T}_{L S} [ t ] ) & ( 52 )\end{matrix}$$

The proportions sf_conf_US and sf_conf_LS are as described above. hx is a heat exchange coefficient between the skin and the deep part of the measurement subject. The heat exchange amount calculation unit 18 can calculate the heat exchange coefficient hx according to Formula (53).

$$\begin{matrix}Math . 51 & \\ h x = min \{ e \times h x 0 + ( 1 - e ) \times hx 0 \times hx {1}^{T [ i ] - T ⌈ 0 ] \frac{{T}_{s k} [ t ] - {T}_{s k} [ 0 ]}{a} + \frac{M E T s [ t ] - 1}{b}} , hx\_max \} & ( 53 )\end{matrix}$$

METs[t] is as described above. a, b, e, hx0, hx1, and hx_max are parameters related to the heat exchange coefficient. The parameters a, b, e, hx0, hx1, and hx_max are known values, and it is sufficient if practical values are used and the parameters a, b, e, hx0, hx1, and hx_max are appropriately set through experiments.

### Calculation of Heat Exchange Amount Q₇ between Trunk and Limbs

Next, the heat exchange amount calculation unit 19 calculates the heat exchange amount Q₇ [W] between the trunk U and the limbs L in the deep part of the measurement subject according to Formula (54) on the basis of the estimated value T_{UC}[t] [° C.] of the temperature of the trunk deep layer UC, the temperature T_{LC}[t] [° C.] of the limb deep layer LC, the estimated value Tₛₖ[t] [° C.] of the average skin temperature, and the estimated value T[t] [C] of the deep body temperature at time t, calculated before Δt by the temperature calculation unit 20 (step S210 in FIG. 9).

$$\begin{matrix}Math . 52 & \\ {Q}_{7} = h c c \times ( {T}_{L C} [ t ] - {T}_{U C} [ t ] ) & ( 54 )\end{matrix}$$

hcc is a heat exchange coefficient between the trunk U and the limbs L in the deep part of the measurement subject. The heat exchange amount calculation unit 19 can calculate the heat exchange coefficient hcc according to Formula (55).

$$\begin{matrix}Math . 53 & \\ h c c = f \times h c c 0 + ( 1 - f ) \times h c c 0 \times hcc\_T \times hcc\_M & ( 55 )\end{matrix}$$

f and hcc0 are parameters related to the heat exchange coefficient. The parameters f and hcc0 are known values, and it is sufficient if practical values are used and the parameters f and hcc0 are appropriately set through experiments. hcc_T is a temperature contribution of the heat exchange coefficient hcc, and hcc_M is a METs contribution of the heat exchange coefficient hcc. The heat exchange amount calculation unit 19 can calculate the temperature contribution hcc_T of the heat exchange coefficient hcc according to Formula (56).

$$\begin{matrix}Math . 54 & \\ hcc\_T = min \{ h c c {1}^{T [ t ] - T [ 0 ] + \frac{{T}_{sk} [ t ] - {T}_{sk} [ 0 ]}{a}} , hcc\_Tmax \} & ( 56 )\end{matrix}$$

hcc_Tmax is a prescribed upper limit value of hcc_T. In addition, the heat exchange amount calculation unit 19 may calculate the METs contribution hcc_M of the heat exchange coefficient hcc according to Formula (57).

$$\begin{matrix}Math . 55 & \\ hcc\_M = min \{ h c c {1}^{\frac{aveMETs [ t ] - 1}{b}} , hcc\_Mmax \} & ( 57 )\end{matrix}$$

hcc_Mmax is a prescribed upper limit value of hcc_M. hcc1 is a parameter related to the heat exchange coefficient. The upper limit values hcc_Tmax and hcc_Mmax and the parameter hcc1 are known values, and it is sufficient if practical values are used and the upper limit values hcc_Tmax and hcc_Mmax and the parameter hcc1 are appropriately set through experiments. aveMETs[t] is a time average value (for example, an average value for six minutes) of Mets. The parameters a and b are as described above.

As described above, it is possible to calculate each of the heat quantities Q_{1,U}, Q_{1,L}, Q_{2,US}, Q_{2,UC}, Q_{2,LS}, Q_{2,LC}, Q_{3,U}, Q_{3,L}, Q_{4,U}, Q_{4,L}, Q₅, Q_{6,U}, Q_{6,L}, and Q₇ with the calorific value calculation unit 13, the metabolic rate calculation unit 14, the heat transfer and heat radiation amount calculation unit 15, the skin transpiration amount calculation unit 16, the exhalation transpiration amount setting unit 17, the heat exchange amount calculation unit 18, and the heat exchange amount calculation unit 19.

The temperature calculation unit 20 calculates an estimated value T_{US}[t+Δt] [° C.] of the temperature of the trunk skin layer US after Δt according to Formula (16) on the basis of the estimated value T_{US}[t] [° C.] of the temperature of the trunk skin layer US of the measurement subject at time t, the metabolic rate Q_{2,US} [W] of the trunk skin layer US, the heat transfer and heat radiation amount Q_{3,U} [W] between the skin and the outside air in the trunk U, the skin transpiration amount Q_{4,U} [W] in the trunk U, and the heat exchange amount Q_{6,U} [W] between the deep part and the skin in the trunk U (step S211 in FIG. 9).

The temperature calculation unit 20 calculates an estimated value T_{UC}[t+Δt] [° C.] of the temperature of the trunk deep layer UC after Δt according to Formula (17) on the basis of the estimated value T_{UC}[t] [° C.] of the temperature of the trunk deep layer UC of the measurement subject at time t, the calorific value Q_{1,U} [W] in the deep layer of the trunk U, the metabolic rate Q_{2,UC} [W] of the trunk deep layer UC, the exhalation transpiration amount Q₅ [W], the heat exchange amount Q_{6,U} [W] between the deep part and the skin of the trunk U, and the heat exchange amount Q₇ [W] between the trunk U and the limbs L in the deep part (step S212 in FIG. 9).

The temperature calculation unit 20 calculates an estimated value T_{LS}[t+Δt] [° C.] of the temperature of the limb skin layer LS after Δt according to Formula (18) on the basis of the estimated value T_{LS}[t] [° C.] of the temperature of the limb skin layer LS of the measurement subject at time t, the metabolic rate Q_{2,LS} [W] of the limb skin layer LS, the heat transfer and heat radiation amount Q_{3,L} [W] between the skin and the outside air in the limbs L, the skin transpiration amount Q_{4,L} [W] in the limbs L, and the heat exchange amount Q_{6,L} [W] between the deep part and the skin in the limbs L (step S213 in FIG. 9).

The temperature calculation unit 20 calculates an estimated value T_{LC}[t+Δt] [° C.] of the temperature of the limb deep layer LC after Δt according to Formula (19) on the basis of the estimated value T_{LC}[t] [° C.] of the temperature of the limb deep layer LC of the measurement subject at time t, the calorific value Q_{1,L} [W] in the deep layer of the limbs L, the metabolic rate Q_{2,LC} [W] of the limb deep layer LC, the heat exchange amount Q_{6,L} [W] between the deep part and the skin in the limbs L, and the heat exchange amount Q₇ [W] between the trunk U and the limbs L in the deep part (step S214 in FIG. 9).

As described above, the estimated values T_{US}[t+Δt], T_{UC}[t+Δt], T_{LS}[t+Δt], and T_{LC}[t+Δt] of the temperature can be sequentially calculated.

Then, the temperature calculation unit 20 calculates an estimated value Tₛₖ[t+Δt] [° C.] of the average skin temperature after Δt according to Formula (20) (step S215 in FIG. 9). In addition, the temperature calculation unit 20 calculates an estimated value T[t+Δt] [° C.] of the deep body temperature after Δt according to Formula (21) (step S216 in FIG. 9).

The sweat amount calculation unit 1a performs the above processing of steps S200 to S216 for each cycle Δt. In the next calculation after Δt, it is sufficient if the processing of steps S200 to S216 is performed by setting T_{US}[t+Δt], T_{UC}[t+Δt], T_{LS}[t+Δt], T_{LC}[t+Δt], Tₛₖ[t+Δt], and T[t+Δt] calculated in the previous time to T_{US}[t], T_{UC}[t], T_{LS}[t], T_{LC}[t], Tₛₖ[t], and T[t], respectively.

Note that in the present example, the case of using the heart rate of the measurement subject has been described, but the pulse rate may be used instead of the heart rate.

The operation of the sweat electrolyte concentration estimation unit 2a of the dehydration estimation device (step S101a in FIG. 6) is the same as that in the second example, and the operations of the blood electrolyte concentration estimation unit 4, the dehydration determination unit 5, the water intake upper limit calculation unit 6, the dehydration predicted time calculation unit 7, and the notification unit 8 (steps S102 to S106 in FIG. 6) are the same as those in the first example.

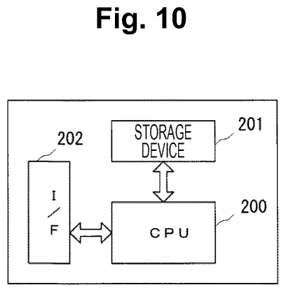

The dehydration estimation device of the first to third examples can be achieved by a computer including a central processing unit (CPU), a storage device, and an interface, and a program controlling these hardware resources. A configuration example of the computer is illustrated in FIG. 10.

The computer includes a CPU 200, a storage device 201, and an interface device (I/F) 202. The I/F 202 is connected to a sensor unit of the sweat amount measurement unit 1, a sensor unit of the sweat electrolyte concentration measurement unit 2, the heart rate measurement unit 10, the temperature measurement unit 11, the humidity measurement unit 12, and hardware of a circuit unit of the notification unit 8, and the like.

In such a computer, a dehydration estimation program for achieving the dehydration estimation method of the first to third examples is provided in a state of being recorded in a recording medium such as a flexible disk, a CD-ROM, a DVD-ROM, or a memory card. The CPU 200 writes a program read from the recording medium into the storage device 201, and executes the processing described in the first to third examples according to the program stored in the storage device 201. The dehydration estimation program can also be provided via a network. In addition, the configuration of the dehydration estimation device may be distributed to a plurality of computer devices.

### INDUSTRIAL APPLICABILITY

The embodiments of the present invention can be applied to a technique for grasping a dehydration state of a person.

### REFERENCE SIGNS LIST

1 Sweat amount measurement unit
1a Sweat amount calculation unit
2 Sweat electrolyte concentration measurement unit
2a Sweat electrolyte concentration estimation unit
3 Storage unit
4 Blood electrolyte concentration estimation unit
5 Dehydration determination unit
6 Water intake upper limit calculation unit
7 Dehydration predicted time calculation unit
8 Notification unit
9 Power supply unit
10 Heart rate measurement unit
11 Temperature measurement unit
12 Humidity measurement unit
13 Calorific value calculation unit
14 Metabolic rate calculation unit
15 Heat transfer and heat radiation amount calculation unit
16 Skin transpiration amount calculation unit
17 Exhalation transpiration amount setting unit
18,19 Heat exchange amount calculation unit
20 Temperature calculation unit
21 Division unit
22 Heat quantity calculation unit
23 Sweat amount estimation unit

1-8. (canceled)

9. A dehydration estimation device comprising:
one or more processors; and
a storage device storing a program to be executed by the one or more processors, the program including instructions for:
estimating a blood electrolyte concentration of a measurement subject; and
determining presence or absence of dehydration of the measurement subject on a basis of the blood electrolyte concentration.

10. The dehydration estimation device according to claim 9, wherein the program further includes instructions for:
measuring a sweat amount of the measurement subject; and
measuring a sweat electrolyte concentration of the measurement subject, wherein the blood electrolyte concentration is estimated on a basis of the sweat amount and the sweat electrolyte concentration.

11. The dehydration estimation device according to claim 9, wherein the program further includes instructions for:
measuring a sweat amount of the measurement subject; and
estimating a sweat electrolyte concentration of the measurement subject on a basis of the sweat amount, wherein the blood electrolyte concentration is estimated on a basis of the sweat amount and the sweat electrolyte concentration.

12. The dehydration estimation device according to claim 9, wherein the program further includes instructions for:
measuring a heart rate of the measurement subject;
measuring a temperature in a vicinity of the measurement subject;
measuring a humidity in a vicinity of the measurement subject;
estimating a sweat amount of the measurement subject on a basis of measurement results of the heart rate, the temperature, and the humidity; and
estimating a sweat electrolyte concentration of the measurement subject on a basis of the sweat amount, wherein the blood electrolyte concentration is estimated on a basis of the sweat amount and the sweat electrolyte concentration.

13. The dehydration estimation device according to claim 12, wherein the program further includes instructions for:
calculating an upper limit value of a normal range of water intake of the measurement subject on a basis of the sweat amount, the sweat electrolyte concentration, and the blood electrolyte concentration; and
predicting a transition of a future blood electrolyte concentration on a basis of the sweat amount, the sweat electrolyte concentration, and the blood electrolyte concentration, and estimate a time until the blood electrolyte concentration reaches the upper limit value of the normal range.

14. The dehydration estimation device according to claim 13, wherein
when the sweat amount is SW[t] and the sweat electrolyte concentration is C_{SW}[t], intracellular fluid water amount V_{IC}[t+Δt] is estimated at time t+Δt after Δt on a basis of an intracellular fluid water amount V_{IC}[t] estimated immediately before for the measurement subject, estimates an extracellular fluid water mount V_{EC}[t+Δt] at time t+Δt on a basis of an extracellular fluid water mount V_{EC}[t] estimated immediately before for the measurement subject, the sweat amount SW[t], and a body surface area of the measurement subject, estimates an osmotic pressure C_{IC}[t+Δt] of an intracellular fluid at time t+Δt on a basis of the intracellular fluid water amount V_{IC}[t], an osmotic pressure C_{IC}[t] of an intracellular fluid estimated immediately before for the measurement subject, and the intracellular fluid water amount V_{IC}[t+Δt], and estimates a blood electrolyte concentration C_{EC}[t+Δt] at time t+Δt on a basis of a blood electrolyte concentration C_{EC}[t] estimated immediately before for the measurement subject, the extracellular fluid water mount V_{EC}[t], the sweat electrolyte concentration C_{SW}[t], the sweat amount SW[t], the body surface area, and the extracellular fluid water mount V_{EC}[t+Δt].

15. A method comprising:
measuring or estimating a sweat amount of a measurement subject;
measuring or estimating a sweat electrolyte concentration of the measurement subject;
estimating a blood electrolyte concentration of the measurement subject on a basis of the sweat amount and the sweat electrolyte concentration; and
determining presence or absence of dehydration of the measurement subject on a basis of the blood electrolyte concentration.

16. A method for estimating dehydration, comprising:
measuring, by a sensor attached to a measurement subject, a sweat amount of the measurement subject;
measuring, by the sensor, an electrolyte concentration in sweat of the measurement subject;
storing time-series data of the sweat amount and the sweat electrolyte concentration;
estimating a blood electrolyte concentration of the measurement subject based on the time-series data of the sweat amount and the sweat electrolyte concentration; and
determining a dehydration state of the measurement subject based on the estimated blood electrolyte concentration.

17. The method of claim 16, wherein the sensor calculates the sweat amount of the measurement subject based on the characteristic of current application between electrodes of the sensor.

18. The method of claim 16, wherein the wearable sensor calculates the electrolyte concentration in sweat of the measurement subject based on light receiving characteristics of a light receiving element of the sensor.
